(19) United States
(12) Patent Application Publication
(10) Pub. No.: US 2015/0202067 A1
(43) Pub. Date: Jul. 23, 2015

(54) DELIVERY DEVICE FOR AN INSERTABLE MEDICAL DEVICE

(71) Applicant: COOK MEDICAL TECHNOLOGIES LLC, Bloomington, IN (US)

(72) Inventors: Zoe Barrand, Greenslopes (AU); Werner D. Ducke, Eight Mile Plains (AU); Adrian Moran, Westmeath (IE); Mena Yang, Browns Plains (AU)

(21) Appl. No.: 14/590,341

(22) Filed: Jan. 6, 2015

(30) Foreign Application Priority Data
Jan. 8, 2014 (AU) 2014200124

(51) Int. Cl. A61F 2/966; A61F 2/07

(52) U.S. Cl. CPC A61F 2/966; A61F 2/07

(57) ABSTRACT

A stent graft and delivery device assembly comprises a dilator 3, a stent graft receiving portion distal of the dilator, an elongate seal-engaging sleeve portion 185 distal of the stent graft receiving portion, a stent graft 35 mounted on the stent graft receiving portion, a stent graft cover 100 mounted around the stent graft, the cover having a maximum outer diameter, and an introducer 40 disposed around the elongate seal-engaging sleeve portion. The introducer has a hollow body, a haemostatic device 33 and an introducer catheter 45 extending from the hollow body. The inner diameter D of the catheter is greater than the outer diameter Y of the seal-engaging sleeve portion 185. The seal-engaging sleeve portion has a maximum outer diameter Y less than the maximum outer diameter C of the cover 100. The introducer catheter is constructed to facilitate blood flow from upstream to downstream of an introduction zone, either by having a plurality of holes therein, or being held in place by an inflatable toroidal seal.

### CROSS REFERENCE TO RELATED APPLICATIONS

This application claims the benefit of Australian patent application No. 201400124 filed on Jan. 8, 2014 entitled A STENT GRAFT AND DELIVERY DEVICE HAVING A LOW PROFILE the entire contents of which are incorporated herein by reference.

### TECHNICAL FIELD

This invention relates to a delivery device for an insertable medical device and to an assembly comprising two such devices. It concerns a method and means for deploying a prosthesis within a body lumen, or guiding interventional devices into a body lumen and in particular to a stent graft and delivery device assembly, especially one having a low profile to reduce the interruption to blood flow. For example the invention may be used for introducing an expandable intraluminal prosthesis (or stent graft) in an endovascular procedure for the repair of diseased or damaged vessels.

### BACKGROUND

The deployment of intraluminal prostheses into the lumen of a patient from a remote location by the use of a deployment device or introducer has been disclosed in a number of earlier patent specifications.

Numerous procedures have been developed that involve the percutaneous insertion of a medical device into a body lumen, such as a blood vessel or duct, of a patient's body. Such a device may be introduced into the lumen by a variety of known techniques. For example, a wire guide may be introduced into a blood vessel using the Seldinger technique. This technique involves creating a surgical opening in the vessel with a needle and inserting a wire guide into the vessel through a bore of the needle. The needle can be withdrawn, leaving the wire guide in place. A delivery device is then inserted over the wire guide and into the vessel. The delivery device may be used in conventional fashion to insert into the blood vessel a variety of medical devices, such as stents, stent grafts, catheters, cardiac leads, balloons, and the like.

For example, the delivery device may be used to deliver and deploy an expandable prosthesis, such as a stent graft, to an aneurysmal blood vessel site. A stent graft is usually formed from a tubular body of a biocompatible graft material with one or more stents mounted into or onto the tubular body to provide support therefor. The stents may be balloon expandable stents and/or self-expanding stents. The deployment of the prosthesis into the lumen of a patient from a remote location by the use of an introducer delivery and deployment device is described in, e.g., U.S. Pat. No. 7,435,253 to Hartley entitled “A Prosthesis and a Method and Means of Deploying a Prosthesis,” which is incorporated herein by reference in its entirety.

Delivery devices are configured to retain a prosthesis in a delivery configuration during delivery to the desired deployment site. The delivery catheter typically includes an inner catheter/cannula spaced from an outer sheath to define a prosthesis retaining region for receiving the prosthesis. The prosthesis is loaded onto an inner cannula along a prosthesis retaining region, with an outer sheath retaining the prosthesis in the delivery configuration. After the delivery device is delivered to the desired deployment site, the prosthesis may be deployed, for example, with retraction of the outer sheath relative to the inner cannula away from the prosthesis to allow for expansion thereof. Accurate placement of an appropriately sized prosthesis should sufficiently cover the target site for treatment and the ends of the prosthesis should be engaged with healthy tissue of the body lumen.

It is desirable to load an appropriately sized prosthesis into a delivery device that is as small as possible. Besides the size of the prosthesis being a factor in the size selection of a delivery device, the shape and size of the body lumen can also be important. Thus, introducing a delivery device that is relatively smaller than the body lumen can avoid potential blockage of fluid or blood flow within the lumen, which can adversely affect the lumen and other parts of the body. Furthermore, the body lumen can be tortuous, thus making relatively smaller delivery devices easier to pass through the tortuous portions.

### SUMMARY

According to a first aspect, there is provided a delivery device assembly comprising a portion for receiving an insertable medical device, a cover mounted around said portion, and an introducer with an introducer catheter arranged to be disposed around the cover and having a proximal end in the form of an insertion portion which has a structure which allows flow past the insertion when inserted in a lumen.

Thus, the introducer catheter is arranged and constructed to facilitate blood flow from upstream of an introduction zone into a vascular system to downstream of the introduction zone.

Preferably, the delivery device further comprises a dilator disposed proximally of the medical device receiving portion, and an elongate seal-engaging sleeve portion disposed distally of the medical device receiving portion, the seal-engaging sleeve portion having an engagable outer diameter, the introducer having a hollow body and a haemostatic device, the introducer catheter extending proximally from the hollow body, the introducer catheter having an inner diameter, the inner diameter being greater than the engagable outer diameter of the elongate seal-engaging sleeve portion, and the elongate seal-engaging sleeve portion having a maximum outer diameter less than a maximum outer diameter of the cover.

Preferably, the haemostatic device comprises at least one seal engageable by the elongate seal-engaging portion and the elongate seal-engaging sleeve portion meets the cover in a transition region.

In one embodiment, the ratio of the inner diameter of the introducer catheter to that of the engagable outer diameter of the elongate seal-engaging sleeve portion is greater than 1.2 to 1.

In one embodiment, the ratio of the inner diameter of the introducer catheter to that of the engagable outer diameter of the elongate seal-engaging sleeve portion is greater than 1.5 to 1.

In one embodiment, the assembly further comprises a dockable device slidably mounted to the seal-engaging sleeve portion, the dockable device having a seal that is adapted to sealing engage the elongate seal-engaging sleeve portion.

In one embodiment, the dockable device is movable from a first position distally adjacent to the stent graft receiving portion but spaced from the introducer to a second position on a distal end of the introducer.

In one embodiment, the dockable device includes an interlocking mechanism that reversibly interlocks the dockable device to the haemostatic device.

In one embodiment, the haemostatic device comprises at least one disk valve.

In one embodiment, the haemostatic device comprises a plurality of disk valves.

In one embodiment, the assembly further comprises a guide wire catheter, the guide wire catheter extending from the dilator to a handle at a distal end of the delivery device, the guide wire catheter slidable over a guide wire.

In one embodiment, the assembly further comprises a sleeve disposed around the guide wire, the sleeve having the stent graft receiving portion at a proximal end thereof and the elongate seal-engaging sleeve portion distal of the stent graft receiving portion.

In one embodiment, the introducer catheter comprises an external tubular wall, the wall having a plurality of holes, the holes arranged to facilitate blood flow from upstream of the introduction zone into a vascular system to downstream of the introduction zone.

In one embodiment, the introducer catheter comprises an external tubular wall and a toroidal seal at a proximal end thereof.

In one embodiment, the toroidal seal is expandable from a deflated condition to an inflated condition.

In one embodiment, the introducer catheter comprises an inflation fluid delivery tube, the inflation fluid delivery tube connected to the toroidal seal.

According to a second aspect of the invention, there is provided a stent graft and delivery device assembly, the assembly comprising: a dilator; a guide wire catheter, the guide wire catheter extending from the dilator to a handle at a distal end of the delivery device, the guide wire catheter slidable over a guide wire; a sleeve disposed around the guide wire catheter, the sleeve having a stent graft receiving portion at a proximal end thereof and a elongate seal-engaging sleeve portion distal of the stent graft receiving portion; a stent graft mounted to the stent graft receiving portion; a stent graft cover mounted around the stent graft, the cover having a maximum outer diameter; and an introducer disposed around the elongate seal-engaging sleeve portion, the introducer having a hollow body, a haemostatic device and an introducer catheter extending proximally from the hollow body, the introducer catheter having an inner diameter, the inner diameter greater than the outer diameter of the elongate seal-engaging sleeve portion, wherein the elongate seal-engaging sleeve portion has a maximum outer diameter less than the maximum outer diameter of the cover, and the elongate seal-engaging sleeve portion meets the cover in a transition region, and, wherein the introducer catheter is arranged and constructed to facilitate blood flow from upstream of an introduction zone into a vascular system to downstream of the introduction zone.

In one embodiment, the ratio of the inner diameter of the introducer catheter to that of the engagable outer diameter of the elongate seal-engaging sleeve portion is greater than 1.2 to 1.

In one embodiment, the ratio of the inner diameter of the introducer catheter to that of the engagable outer diameter of the elongate seal-engaging sleeve portion is greater than 1.5 to 1.

In one embodiment, the assembly further comprises a dockable device slidably mounted to the seal-engaging sleeve portion, the dockable device having a seal that is adapted to sealing engage the elongate seal-engaging surface.

### BRIEF DESCRIPTION OF DRAWINGS

Embodiments of the present invention will be discussed with reference to the accompanying drawings wherein:

FIG. 1 is an isometric view of part of a stent graft and delivery device assembly according to an embodiment of the invention;

FIG. 2 is an isometric view of an introducer, the introducer being part of the delivery device assembly shown in FIG. 1;

FIG. 3 is a generalised isometric view showing components of the stent graft and delivery device assembly of FIGS. 1 and 2;

FIG. 4A is a side view showing parts of the delivery device of FIG. 1;

FIGS. 4B and 4C are cross-sectional views through section lines B-B and C-C, as shown on FIG. 4A;

FIG. 5A is a diagrammatic view showing an introducer catheter of the introducer of FIG. 2 inserted into a femoral artery;

FIG. 5B is a close up view of the distal end of the introducer catheter shown in FIG. 5A;

FIG. 6 is a similar view to that of FIG. 3 but shows an alternative introducer;

FIG. 7 is a similar view to that of FIG. 2 but shows the alternative introducer of FIG. 6 in more detail;

FIGS. 8A and 8B show the introducer of FIGS. 6 and 7 in a deflated and inflated condition within a femoral artery respectively;

FIGS. 9A and 9B are detailed views of a portion of the alternative introducer shown in FIGS. 12A and 12B;

FIGS. 10A to 10E are diagrammatic views showing deployment of the delivery device according to the invention into an arterial system of a patient.

FIGS. 11 and 12 are isometric and cross-sectional views respectively of a dockable device being a component of the stent graft and the delivery device assembly shown in FIG. 3;

FIG. 13 is a cross-sectional view showing the delivery device of the invention passing through a haemostatic device of the invention;

FIG. 14 is a similar view to that of 13 but shows the dockable device of FIGS. 11 and 12 docked with the haemostatic device;

FIGS. 15 and 16 show an alternative embodiment of the dockable device shown in FIGS. 11 and 12 in isometric and cross-sectional views respectively; and

FIG. 17 shows the dockable device of FIGS. 15 and 16 docked to the haemostatic device of FIG. 13.

### DESCRIPTION OF EMBODIMENTS

For the purpose of understanding the principles of the invention, reference will now be made to the embodiments illustrated in the drawings, and specific language will be used to describe the same. It is to be understood that the Figures are schematic and do not show the various components in their actual scale. In many instances, the Figures show scaled up components to assist the reader.

Throughout this specification, the term distal with respect to a portion of the aorta, a deployment device or an endograft means the end of the aorta, deployment device or endograft further away in the direction of blood flow away from the heart and the term proximal means the portion of the aorta, deployment device or end of the endograft nearer to the heart. When applied to other vessels, similar terms such as caudal and cranial should be understood.

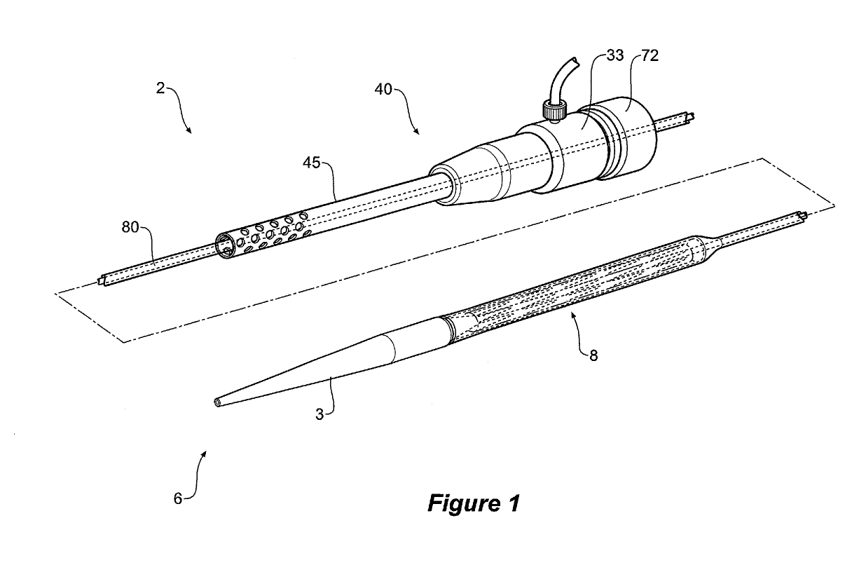

Referring now to FIG. 1, there is shown a delivery device assembly for the percutaneous insertion into the artery (or other bodily lumen) of medical devices such as stents, stent grafts, catheters, cardiac leads, balloons, and the like. With the embodiment shown in FIG. 1, the delivery device assembly is loaded with a stent graft.

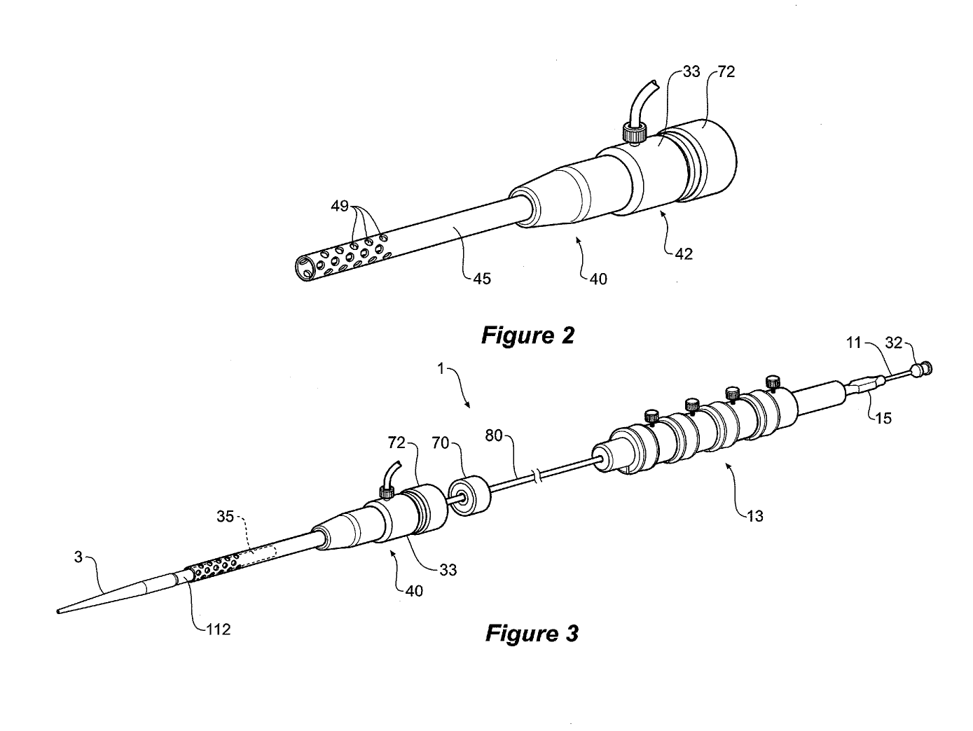

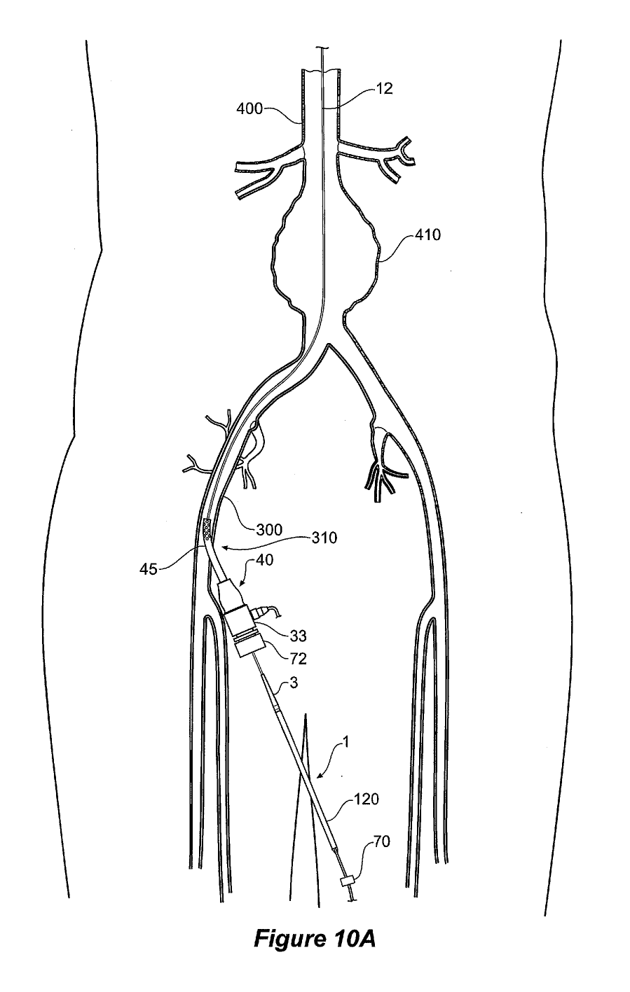

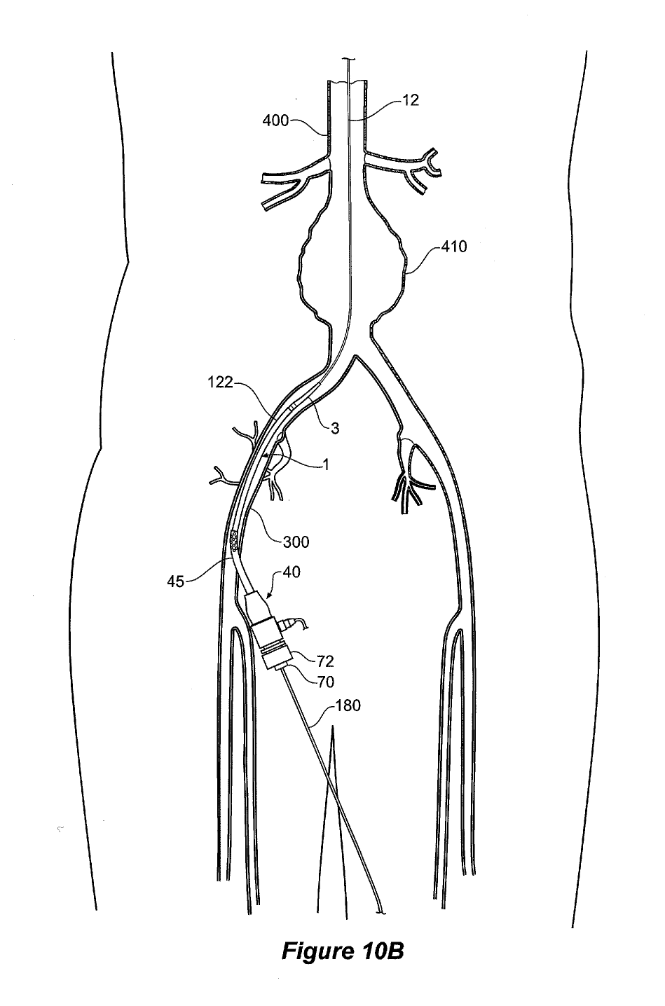

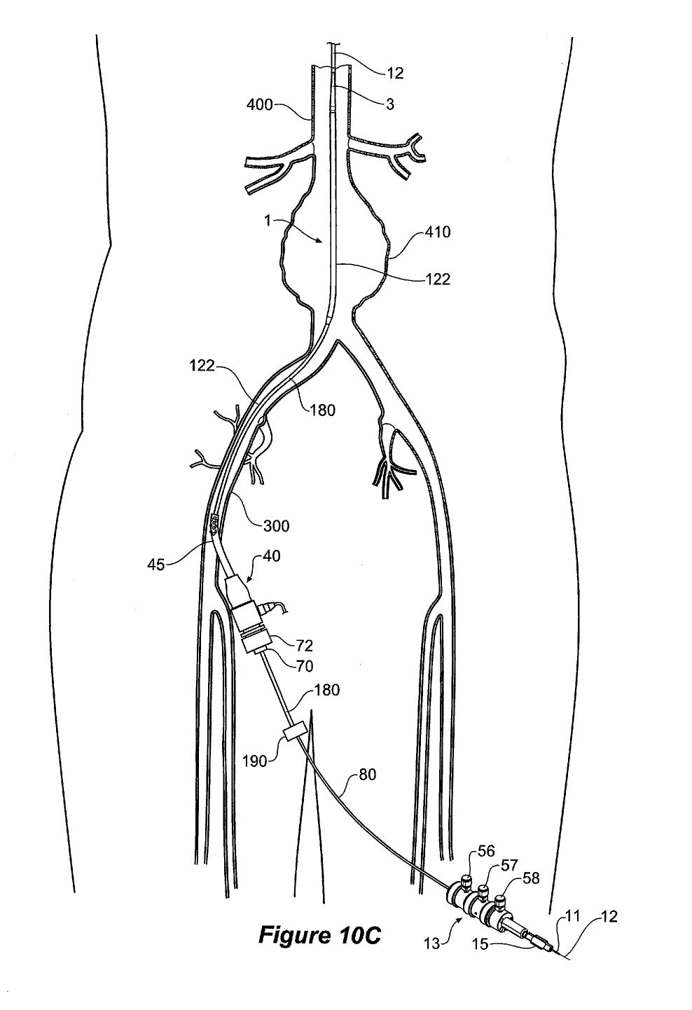

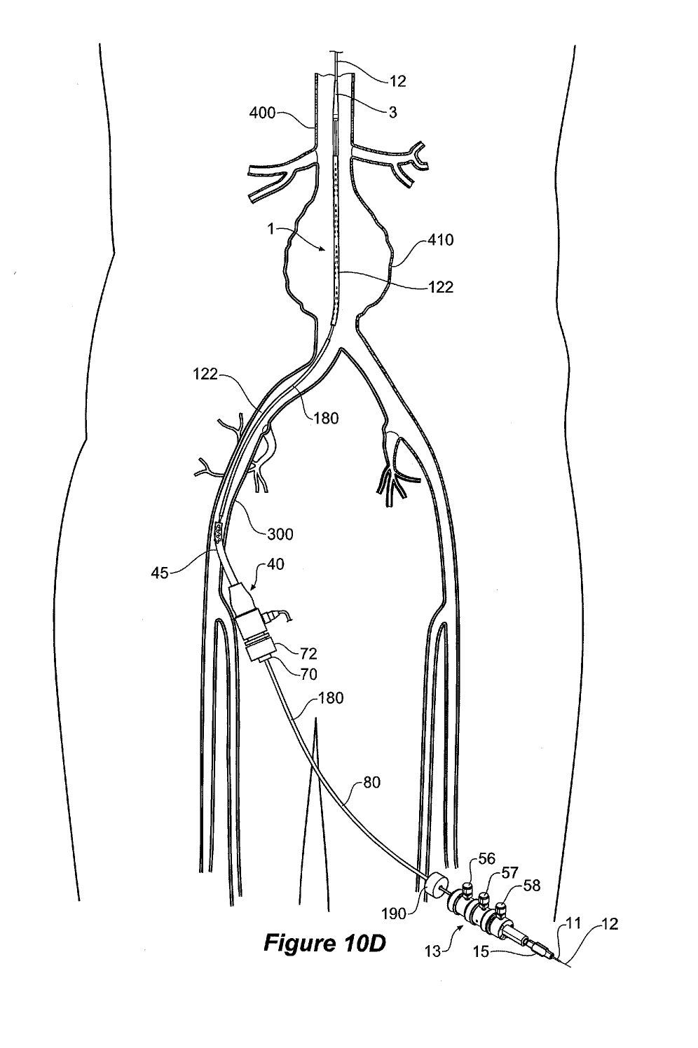

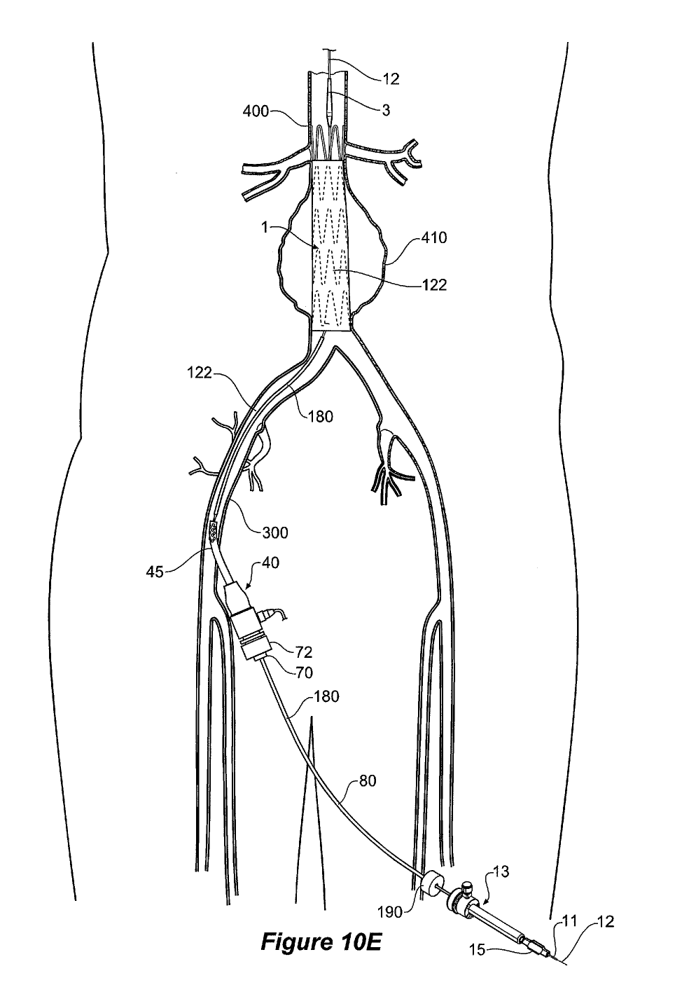

The delivery device assembly has two distinct components. The first component, an introducer 40, is shown in FIG. 2. The introducer 40 has a tubular introducer catheter 45 for insertion over a guide wire into a bodily lumen such as a femoral artery, for instance, as is shown in FIG. 10A. The introducer 40 includes a haemostatic device 33 for controlling blood loss through the delivery device. The haemostatic device 33 is fixedly connected to the introducer catheter 45. The haemostatic device 33 comprises one or more haemostatic valves or seals. Suitable haemostatic valves include, for example, disk/disc valves, iris valves, and the like that provide a seal.

The second component of the assembly is a delivery device 2 as shown in FIG. 3 passing through the introducer 40. As can be seen from FIGS. 1, 3 and 10A, the delivery device 2 includes a dilator 3 at a proximal end thereof. Adjacent the dilator and distal of the dilator 3 is a stent graft receiving portion 8 with a stent graft 35 mounted in the receiving portion. At a distal end of the delivery device 2 is a connection means 32 in the form of a Luer lock connector (adapted to accept a syringe for introduction of reagents).

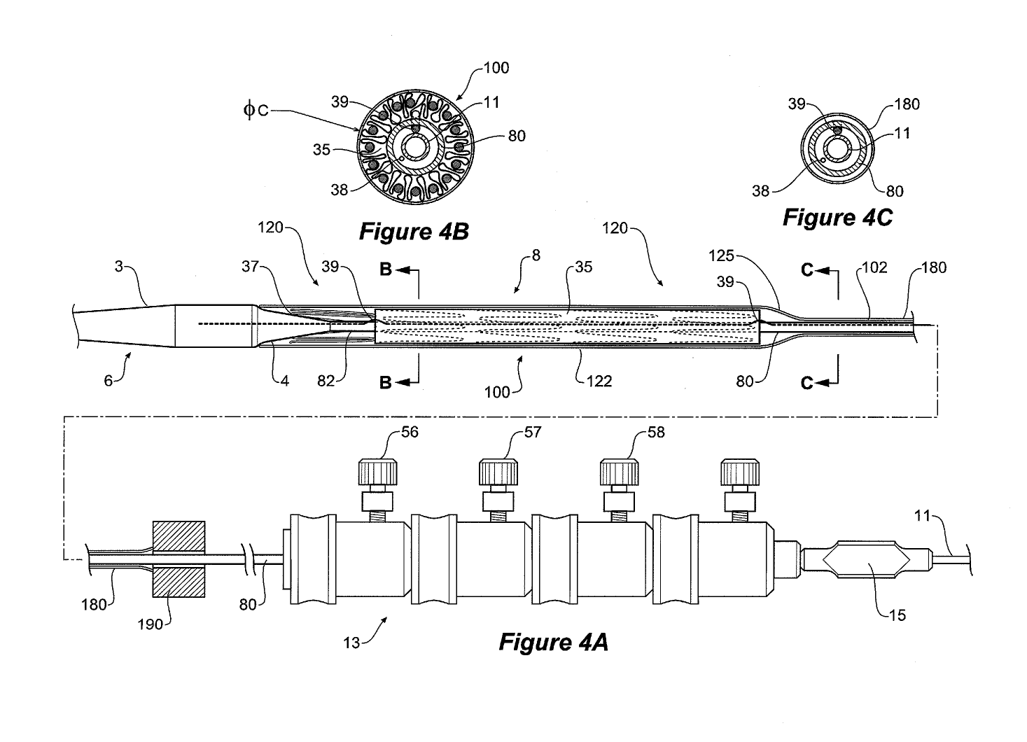

FIG. 4A shows the proximal end 6 of the delivery device assembly 1, the stent graft receiving portion 8 and its associated stent graft 35 in more detail. FIG. 4A shows a stent graft cover 122 in the form of a thin cover 100.

Also shown in FIG. 4A is a guide wire catheter 11. The guide wire catheter 11 extends from the dilator 3 to a handle 13 at a distal end of the delivery device 2. The guide wire catheter 11 is slidable over a guide wire, such as the guide wire 12 illustrated in FIG. 10A.

Still referring to FIG. 4A, the cover 122 transitions at transition portion 125 into an elongate seal-engaging sleeve portion 180 that extends right though the introducer 40, and its haemostatic device 33, and out of its distal end external to the patient's body. A puller 190 is provided so as to actuate the removal of the cover 122. This seal-engaging sleeve portion 180 engages a seal within a haemostatic device 33 that forms part of the introducer 40 shown in FIG. 2. More specifically, the engagable maximum outer diameter of the seal-engaging sleeve portion 180 is engaged by the disk valves or seals 33′ of the haemostatic device 33.

Turning now to FIG. 4B, which is a cross-sectional view through section lines B-B on FIG. 4A, the stent graft 35 can be seen disposed between the cover 100 and the guide wire catheter 11. A guide wire catheter sleeve 80 between the guide wire catheter 11 and the stent graft 35 may also be provided, as is shown in FIGS. 4A-4C.

Turning now to FIG. 4C, inboard of the sleeve 80 is a space formed between the inside of the sleeve 80 and the outside of the guide wire catheter 11. This space accommodates a stent graft end release wire 39 (or two stent graft end release wires—one for a proximal end and one for a distal end) and a stent graft compression release wire 38. The stent graft end release wire 39 exits the sleeve 80 in the two positions shown in FIG. 4A so as to releasably retain the proximal and distal ends of the stent graft 80, as is known in the art. The stent graft compression release wire 38 also exits the sleeve 80 so as to selectively retain and release the stent graft using diameter releasing ties or other methods known in the art.

A cross-sectional view of FIG. 4C, taken through section lines C-C, show that the overall diameter of the delivery device 2 reduces in a direction distal of the stent graft receiving portion 8 of the delivery device 2. The reduced overall diameter assists in minimising the interruption to blood flow that may otherwise be caused by larger diameter delivery devices.

The stent graft cover 100 of the invention may take many forms. With the form shown in FIGS. 1, 3, 4A, 4B and 4C, the cover 100 is part of an outermost sheath 120. The outermost sheath 120 has a cover 100 that transitions an elongate seal-engaging sleeve portion 180 through a transition portion 125, as is most clearly shown in FIG. 4A. The outermost sheath 120 may be made from various suitable materials. It may be made from a material that holds its general shape to the shape shown in the figures. Alternatively, it may be made from a stretchable, resilient material that stretches over the loaded stent graft 35 in the cover region 100.

In other embodiments of the invention, the cover 100 may be a membrane 100 that only covers the stent graft 35 and terminates just distally of the stent graft 35 that it is covering. Such a membrane 100 may be retractable into the guide wire catheter sleeve 80, or may be dissolvable, for instance. To facilitate retraction of such a membrane 100, the membrane 100 may be frangible along longitudinal lines. With such an arrangement, the puller 190 described above with reference to FIG. 4A is either not required, or takes a different form.

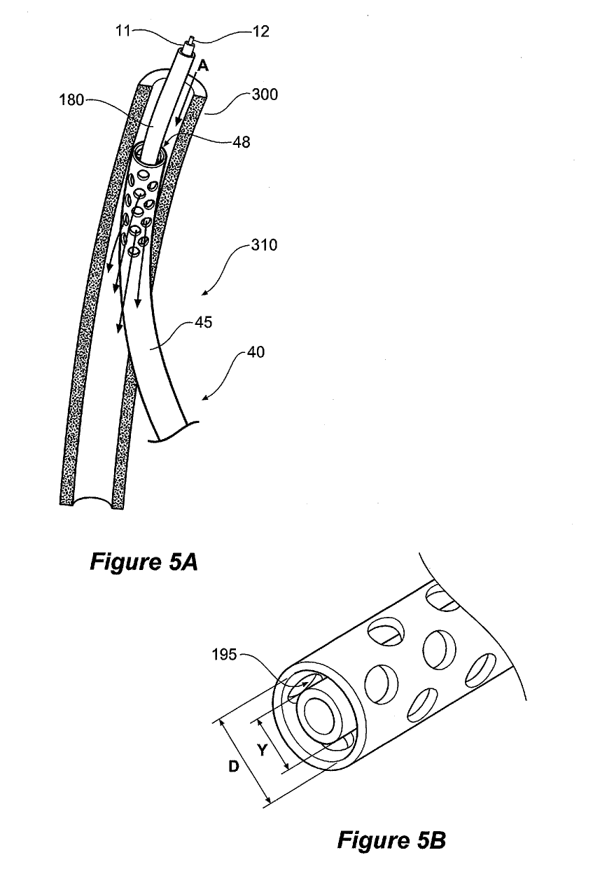

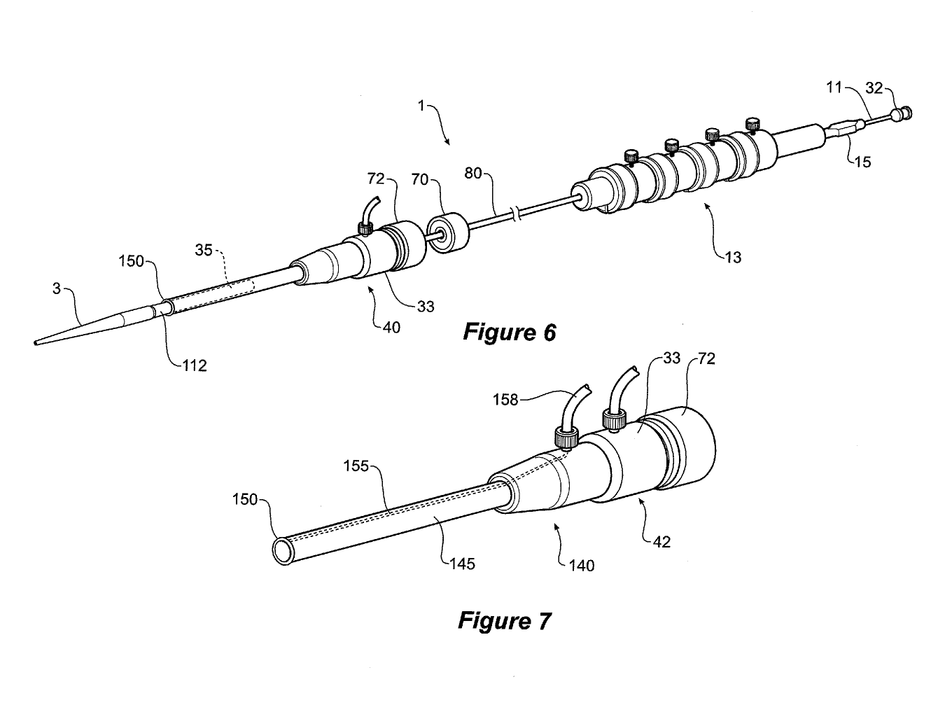

The introducer 40 disposed around the sleeve 80 and valve-engaging sleeve portion 180 is more clearly seen in FIG. 5A. The tubular introducer catheter 45 of the introducer 40 has an inner diameter D, the inner diameter D greater than the engagable outer diameter Y of the elongate seal-engaging sleeve portion 185, as is illustrated in FIG. 5B. This diameter difference forms an annular lumen 195 through which blood may flow.

The elongate seal-engaging sleeve portion 180 has an engagable maximum outer diameter Y that is less than the maximum outer diameter C of the cover 100, as is illustrated in FIG. 4B read together with FIGS. 5A and 5B.

The ratio of the inner diameter D of the introducer catheter 45 to that of the outer diameter Y of the elongate seal-engaging sleeve portion 180 is greater than 1.2 to 1. In fact, with the embodiment shown in the Figures, the ratio is greater than 1.5 to 1. As this ratio increases, the extent to which the delivery device 2 impedes the blood flow is reduced.

As can be seen in FIG. 5A, the tubular introducer catheter 45 of the introducer 40 takes up a considerable portion of the femoral artery 300. With conventional delivery devices, the tubular introducer catheter 45 extends a longer distance up the femoral artery 300 and into the aorta. This significantly reduces blood flow through the femoral artery 300 at and proximally of the introduction zone 310. With embodiments of the present invention, blood flow in the direction indicated by arrow A on FIG. 5A is less interrupted. This is because the blood may flow into the mouth 48 of the introducer catheter 45 and flow along the aforementioned annular lumen 195 and out through apertures 49.

Referring again to FIG. 3, a dockable device 70 and a dock 72 are shown. The thickest part of the delivery device 2 is the nose cone dilator 3 and the adjacent stent graft cover portion 122 which need to pass through the haemostatic device 33 of the introducer 40 as occurs between the positions shown in FIGS. 10A and 10B. The dockable device 70 assists with providing a more effective seal around the smaller diameter elongate seal-engaging sleeve portion 180.

The dockable device 70 has a tight seal provided by a seal 78, in the form of an O-ring for instance, which is sized to accommodate the elongate seal-engaging sleeve portion 180. Other appropriate types of seals instead of O-ring-type seals may also be used. The dockable device 70 docks into the dock 72.

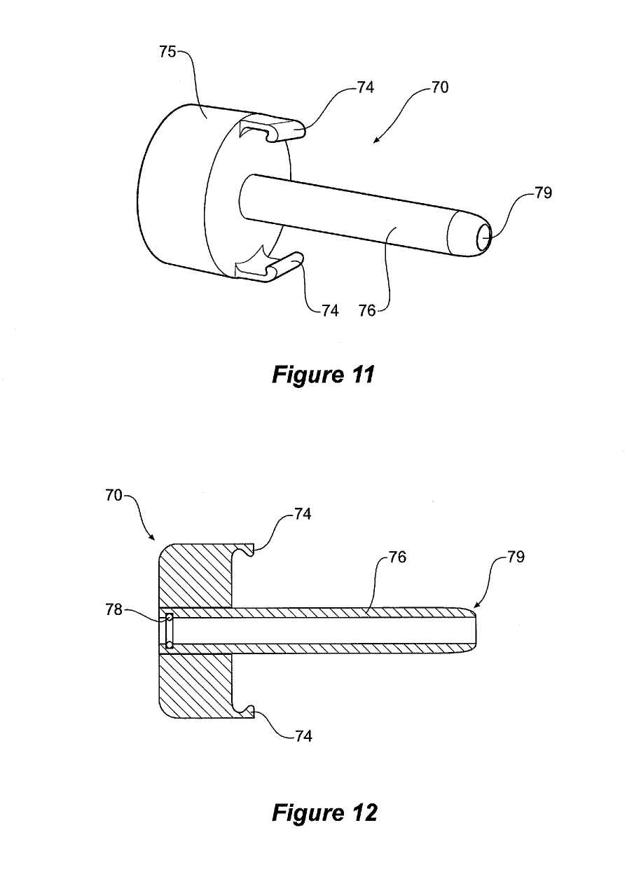

Referring to FIGS. 11, 12, 13 and 14, the dockable device 70 will be described in more detail. The dockable device 70 is slidably mounted to the seal-engaging sleeve portion 180. An annular seal 78, as is most clearly shown in FIG. 12, is provided to seal against the elongate. More specifically, the engagable maximum outer diameter Y of the seal-engaging sleeve portion 180 is engaged by the annular seal 78.

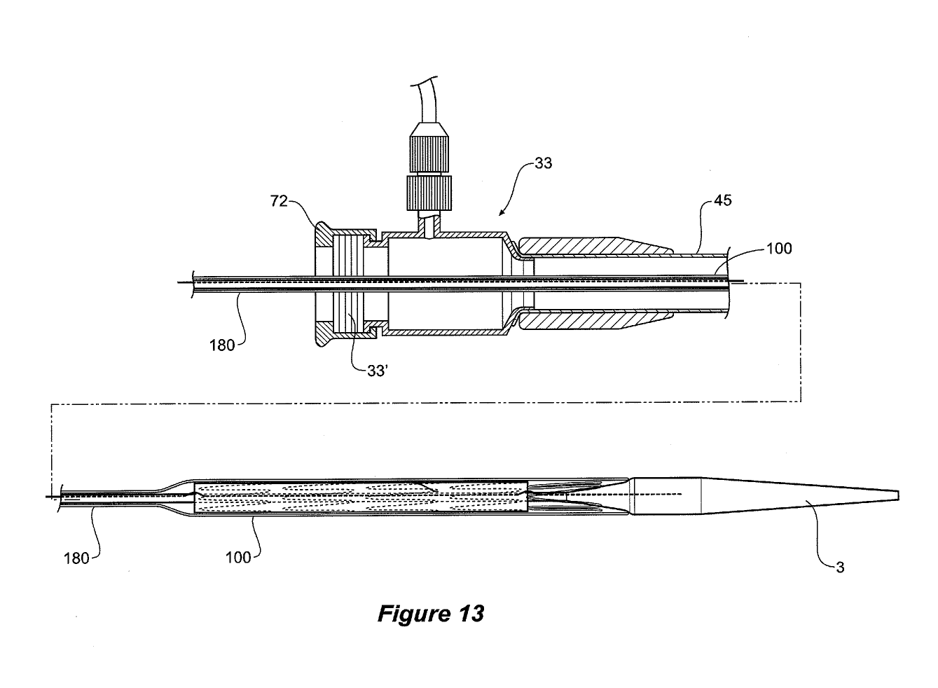

The dockable device 70 includes a body 75 and a sleeve 76. Interlocking mechanisms 74 are provided to reversibly interlock the dockable device 70 to the haemostatic device 33 at dock 72 (as illustrated in FIG. 13).

The aforementioned dockable device 70 is similar to the dockable device described in the applicant's earlier U.S. Pat. No. 8,419,783, which is hereby incorporated by reference.

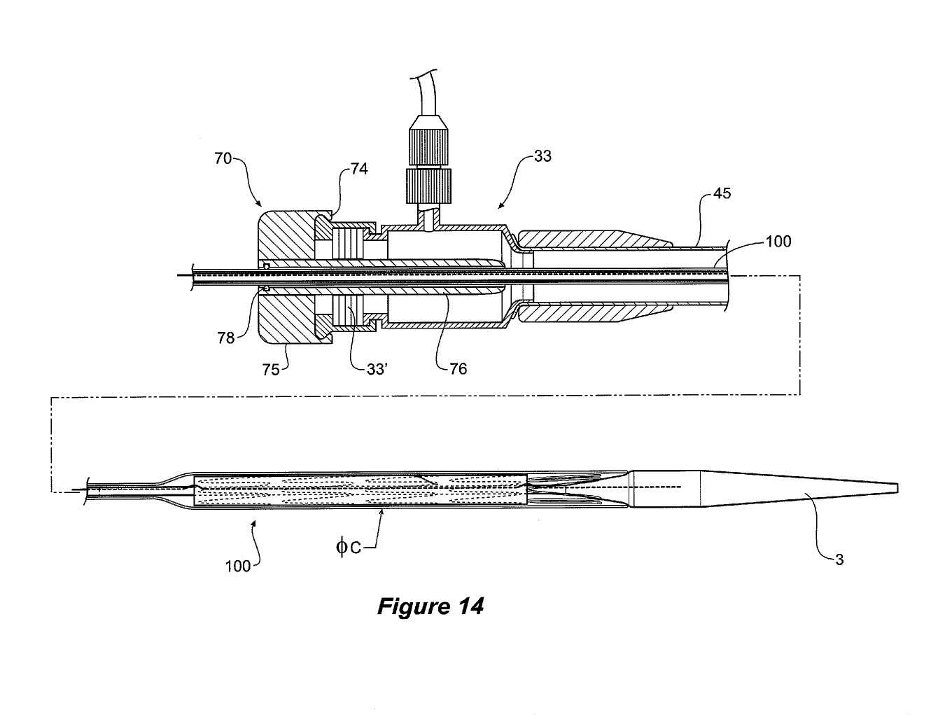

With the dockable device 70 shown in FIGS. 11, 12 and 14, the sleeve 76 has an outer diameter that is similar to or the same as the maximum outer diameter D of the stent graft cover 100. This helps ensure a good seal through the disc valves 33′, as is illustrated in FIG. 14.

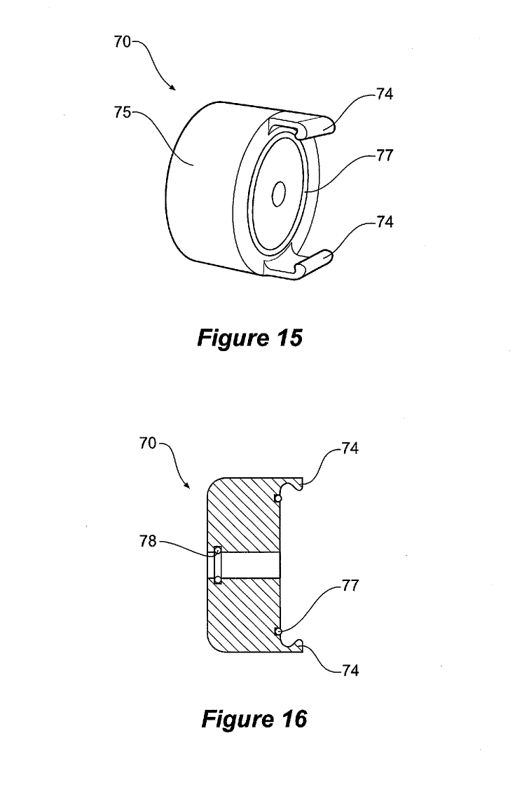

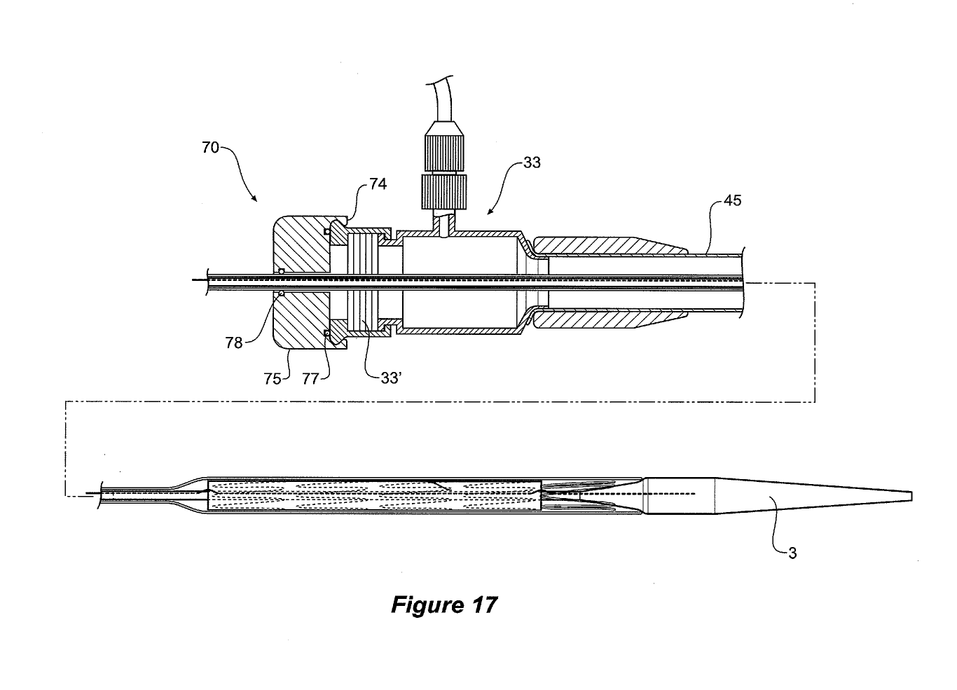

An alternative dockable device 70 is shown in FIGS. 15, 16 and 17. With this version, there is no sleeve 76. An O-ring seal 77 may be provided so as to seal the body 75 against the dock 72. This O-ring seal 77 is not essential and may be deleted in some embodiments as the disc valves 33′ provide sealing. However, the additional sealing may be helpful in some applications.

The guide wire catheter 11 may comprise any suitable biocompatible material or materials including metal or plastic. Suitable materials include, but are not limited to aluminium, nitinol, nylon, polypropylene, and polyethylene. The guide wire catheter sleeve 80 preferably comprises a flexible material that is able to bend and flex to negotiate complex and tortuous inner body lumina. The sleeve 80 may comprise a biocompatible plastic such as PTFE, PEEK, polyethylene, nylon, or the like.

The guide wire catheter sleeve 80, or the guide wire catheter sleeve 80 in combination with the guide wire catheter 11, are constructed to have sufficient longitudinal column strength to ensure adequate pushing force manipulation during delivery and deployment of the stent graft 35.

FIGS. 10A-10E illustrates an example of use of the delivery device 2 deploying a medical device, such as the stent graft 35, into an artery.

The delivery device 2 may be used to percutaneously insert into the artery (or other bodily lumen) a variety of medical devices, such as stents, stent grafts, catheters, cardiac leads, balloons, prostheses, and the like.

A wire guide may be introduced into an artery using the Seldinger technique. This technique involves creating a surgical opening in artery with a needle and inserting a wire guide into the artery through a bore of the needle. The needle can be withdrawn, leaving the wire guide in place.

In FIG. 10A, the tubular introducer catheter 45 of the introducer 40 is inserted over a guide wire 12 into the femoral artery 300 at an introduction zone 310. At this point, the nose cone 3 of the delivery device 2 is distal of the introducer 40. Even further distally is the dockable device 70.

From the position shown in FIG. 10A, the delivery device 2 is advanced over the guide wire 12 to the position shown in FIG. 10B. As can be seen in FIG. 10B, the thickest part of the delivery device 2 has passed through the haemostatic device 33 of the introducer 40. In this position, the elongate seal-engaging sleeve portion 180 is engaged with the disk seal 33 within the haemostatic device 33. The dockable device 70, as for instance shown in FIGS. 15 and 16, assists with providing a more effective seal around the smaller diameter elongate seal-engaging sleeve portion 180, as has been described above.

The docking of the dockable device to the dock 72 may be achieved by means such as a common bayonet or a sleeve with locking screw, or screw thread, or push clip, or any other appropriate means. FIGS. 15 and 16 show a clipable interlocking mechanism 74.

Turning now to FIG. 10C, the delivery device 2 has been advanced proximally to its approximate deployment position within aneurysmal portion 410 of the aorta 400. In this Figure, the handle 13 is now visible. The next step in the deployment procedure is the retraction of the stent graft cover portion 122 of the outermost sheath 120. This step will vary depending on the embodiment of the invention being used.

With the embodiment of the invention shown in FIGS. 4A-4C, sliding the puller 190 at the distal end of the outermost sheath 120 distally to the position shown in FIG. 10D retracts the cover portion 122 of the outermost sheath 120. In this position, it can be seen that the stent graft cover portion 122 of the outermost sheath 120 is now distal of the stent graft 35.

With the stent graft 35 now uncovered, the diameter reducing ties locking screw 58 is removed and the stent graft compression release wire 38 is withdrawn. This allows the stents of the stent graft 35 to expand.

After the stent graft 35 has expanded, the locking screws 56 and 57 are released and the stent graft ends release wires are removed. This frees the proximal and distal ends of the stent graft 35 from the delivery device 2, allowing the stent graft 35 to adopt its final position, as shown in FIG. 10E.

In other embodiments of the invention, a single stent graft end release wire 39 may be used with a single locking screw 37. With such an embodiment, the stent graft end release wire 39 (such as is shown in FIG. 4A for instance), can be progressively withdrawn so as to first release the proximal end of the stent graft 35 and subsequently the distal end of the stent graft 35.

After deployment of the stent graft 35, the delivery device 2 can be progressively removed. The introducer 40 of the delivery device 2 may remain in its position shown in FIGS. 10A to 10E, so as to permit the subsequent passage of other medical devices therethrough into the arterial system.

The delivery device 2 as described above includes an introducer 40 having a tubular introducer catheter 45 with a plurality of apertures 49 provided to facilitate blood flow past the delivery device 2.

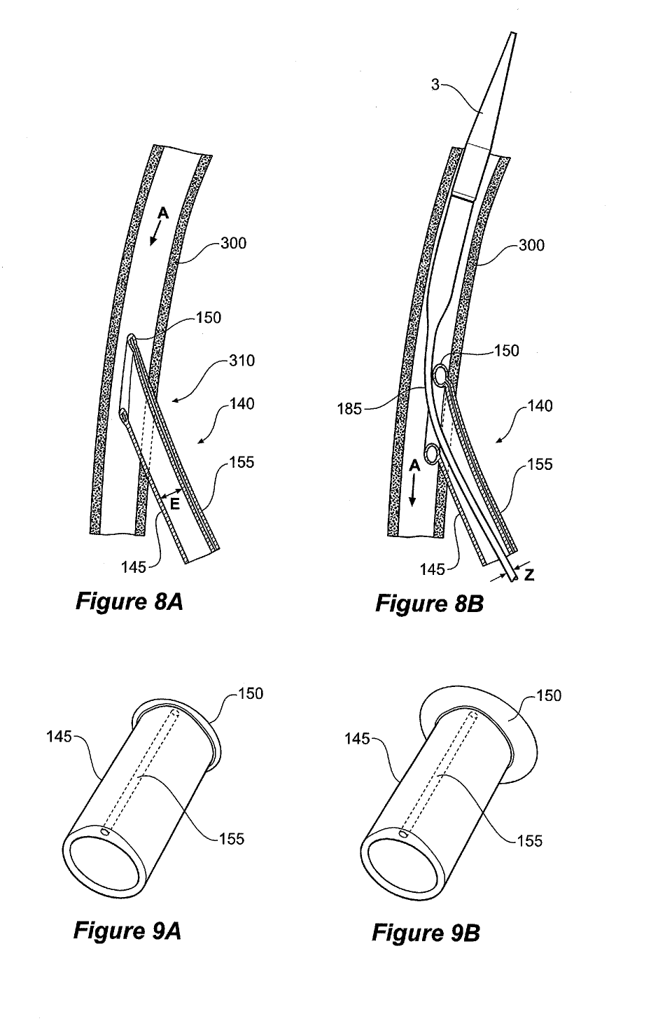

An alternative introducer 140 is shown in FIGS. 7, 8A, 8B, 9A and 9B. The alternative introducer 140 has the same distal end with a haemostatic device 33 and dockable device 70 but has an alternative tubular introducer catheter 145. The alternative introducer catheter 145 terminates with an inflatable seal 150. FIGS. 8A and 9A show the inflatable seal 150 in a deflated condition.

The introducer 140 is inserted over a guide wire 12 into the femoral artery 300 in a similar way to the introducer 140, as described above. This insertion is done with the seal 150 deflated. Once in the position illustrated in FIG. 8A, the inflatable seal 150 is inflated by delivery of a fluid (for instance, sterile saline) via a delivery line 155. The delivery line 155, as shown in FIGS. 9A and 9B, may be integrated into the introducer catheter 145 or may be a separate line. An inlet port 158 such as is shown in FIG. 7 may be provided to facilitate fluid delivery.

Once the inflatable seal 150 has been inflated, the introducer 140 may be slightly retracted from the position shown in FIG. 8A to the position shown in FIG. 8B. The inner diameter E is greater than the outer diameter Z of the valve-engaging external surface 80. This, in combination with the introducer 140 shape and positioning facilitates blood flow in the direction of Arrow A past the insertion point of the introducer 140.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosures in Australian patent application number 20144200124, from which this application claims priority, and in the abstract accompanying this application are incorporated herein by reference.

1. A delivery device assembly comprising:
a portion for receiving an insertable medical device;
a cover mounted around said portion; and
an introducer with an introducer catheter arranged to be disposed around the cover and having a proximal end in the form of an insertion portion which has a structure which allows flow past the insertion when inserted in a lumen.

2. A delivery device according to claim 1 further comprising a dilator disposed proximally of the medical device receiving portion, and an elongate seal-engaging sleeve portion disposed distally of the medical device receiving portion, the seal-engaging sleeve portion having an engagable outer diameter, the introducer having a hollow body and a haemostatic device, the introducer catheter extending proximally from the hollow body, the introducer catheter having an inner diameter, the inner diameter being greater than the engagable outer diameter of the elongate seal-engaging sleeve portion, and
the elongate seal-engaging sleeve portion having a maximum outer diameter less than a maximum outer diameter of the cover.

3. A delivery device according to claim 2 wherein the haemostatic device comprises at least one seal engageable by the elongate seal-engaging portion and the elongate seal-engaging sleeve portion meets the cover in a transition region.

4. A delivery device according to claim 2 wherein the ratio of the inner diameter of the introducer catheter to that of the engagable outer diameter of the elongate seal-engaging sleeve portion is greater than 1.2 to 1.

5. A delivery device according to claim 2 comprising a dockable device slidably mounted to the seal-engaging sleeve portion, the dockable device having a seal that is adapted to sealing engage the elongate seal-engaging sleeve portion.

6. A delivery device according to claim 5 wherein the dockable device is movable from a first position distally adjacent to the stent graft receiving portion but spaced from the introducer to a second position on a distal end of the introducer.

7. A delivery device according to claim 6 wherein the dockable device includes an interlocking mechanism that reversibly interlocks the dockable device to the haemostatic device.

8. A delivery device according to claim 2 comprising a guide wire catheter, the guide wire catheter extending from the dilator to a handle at a distal end of the delivery device, the guide wire catheter being slidable over a guide wire.

9. A delivery device according to claim 8 comprising a sleeve disposed around the guide wire, the sleeve having the stent graft receiving portion at a proximal end thereof and the elongate seal-engaging sleeve portion distal of the stent graft receiving portion.

10. A delivery device according to claim 1 wherein the introducer catheter comprises an external tubular wall, the wall having a plurality of holes, the holes being arranged to facilitate blood flow from upstream of the introduction zone into a vascular system to downstream of the introduction zone.

11. A delivery device according to claim 1 wherein the introducer catheter comprises an external tubular wall and a toroidal seal at a proximal end thereof.

12. A delivery device according to claim 13 wherein the toroidal seal is expandable from a deflated condition to an inflated condition.

13. A delivery device according to claim 14 wherein the introducer catheter comprises an inflation fluid delivery tube, the inflation fluid delivery tube connected to the toroidal seal.

14. A stent graft and delivery device assembly, the assembly comprising:
a dilator;
a guide wire catheter, the guide wire catheter extending from the dilator to a handle at a distal end of the delivery device, the guide wire catheter slidable over a guide wire;
a sleeve disposed around the guide wire catheter, the sleeve having a stent graft receiving portion at a proximal end thereof and a elongate seal-engaging sleeve portion distal of the stent graft receiving portion;
a stent graft mounted to the stent graft receiving portion;
a stent graft cover mounted around the stent graft, the cover having a maximum outer diameter; and
an introducer disposed around the elongate seal-engaging sleeve portion, the introducer having a hollow body, a haemostatic device and an introducer catheter extending proximally from the hollow body, the introducer catheter having an inner diameter, the inner diameter greater than the outer diameter of the elongate seal-engaging sleeve portion,
wherein the elongate seal-engaging sleeve portion has a maximum outer diameter less than the maximum outer diameter of the cover, and the elongate seal-engaging sleeve portion meets the cover in a transition region, and,
wherein the introducer catheter is arranged and constructed to facilitate blood flow from upstream of an introduction zone into a vascular system to downstream of the introduction zone.

15. The assembly of claim 14 comprising a dockable device slidably mounted to the seal-engaging sleeve portion, the dockable device having a seal that is adapted to sealingly engage the elongate seal-engaging surface.

16. The assembly of claim 14 wherein the ratio of the inner diameter of the introducer catheter to that of the engagable outer diameter of the elongate seal-engaging sleeve portion is greater than 1.2 to 1.

17. A stent graft and delivery device assembly comprising:
a dilator;
a stent graft receiving portion distal of the dilator;
an elongate seal-engaging sleeve portion distal of the stent graft receiving portion, the seal-engaging sleeve portion having an engagable outer diameter;
a stent graft mounted to the stent graft receiving portion;
a stent graft cover mounted around the stent graft, the cover having a maximum outer diameter; and;
an introducer disposed around the elongate seal-engaging sleeve portion, the introducer having a hollow body, a haemostatic device and an introducer catheter extending proximally from the hollow body, the introducer catheter having an inner diameter, the inner diameter greater than the engagable outer diameter of the elongate seal-engaging sleeve portion, the haemostatic device comprising at least one seal engagable by the elongate seal-engaging sleeve portion,
wherein the elongate seal-engaging sleeve portion has a maximum outer diameter less than the maximum outer diameter of the cover, and the elongate seal-engaging sleeve portion meets the cover in a transition region, and,
wherein the introducer catheter is arranged and constructed to facilitate blood flow from upstream of an introduction zone into a vascular system to downstream of the introduction zone.

18. The assembly of claim 17 wherein the haemostatic device comprises a plurality of disk valves.

19. The assembly of claim 17 wherein the introducer catheter comprises an external tubular wall, the wall having a plurality of holes, the holes arranged to facilitate blood flow from upstream of the introduction zone into a vascular system to downstream of the introduction zone.

20. The assembly of claim 17 wherein the introducer catheter comprises an external tubular wall and a toroidal seal at a proximal end thereof, wherein the toroidal seal is expandable from a deflated condition to an inflated condition.
